(19) United States
(12) Patent Application Publication
(10) Pub. No.: US 2026/0202298 A1
(43) Pub. Date: Jul. 16, 2026

(54) HIGH-THROUGHPUT MEASUREMENT OF PLEOMORPHIC VIRUS PARTICLE COUNTS, DISTRIBUTIONS, AND COMPOSITION

(71) Applicant: Brandeis University, Waltham, MA (US)

(72) Inventor: Tijana Ivanovic, Lexington, MA (US)

(73) Assignee: Brandeis University, Waltham, MA (US)

(21) Appl. No.: 18/686,261

(22) PCT Filed: Aug. 31, 2022
(86) PCT No.: PCT/US2022/042125

§ 371 Date: Feb. 23, 2024

Related U.S. Application Data
(60) Provisional application No. 63/238,934, filed on Aug. 31, 2021.

(51) Int. Cl. G01N 15/0205; G01N 15/00; G01N 33/569; G01N 33/58

(52) U.S. Cl. CPC G01N 15/0205; G01N 33/56983; G01N 33/582; G01N 2015/0038; G01N 2333/11; G01N 2469/10; G01N 2500/04

(57) ABSTRACT

Described herein is a method of determining a distribution and/or quantification of pleomorphic virus particles in a sample including detecting the pleomorphic virus particles, and determining, based on the detecting, the distribution and/or quantification of spherical virus particles and/or filamentous virus particles in the sample. Also included are methods of screening a test dmg including adding a test drug to a sample comprising pleomorphic virus particles and detecting the distribution and/or quantification of spherical virus particles and/or filamentous virus particles in the sample containing the test drug.

### CROSS-REFERENCE TO RELATED APPLICATIONS

This application claims priority to U.S. Provisional Application 63/238,934 filed on Aug. 31, 2021, which is incorporated herein by reference in its entirety.

### STATEMENT REGARDING FEDERALLY SPONSORED RESEARCH & DEVELOPMENT

This invention was made with government support under GM128204 awarded by the National Institutes of Health. The government has certain rights in the invention.

### FIELD OF THE DISCLOSURE

The present disclosure is related to methods of determining a size/shape distribution and/or quantification of pleomorphic virus particles in a sample, and methods of screening test drugs for their effects on the distribution and/or quantification of pleomorphic virus particles.

### BACKGROUND

Pleomorphic virus particles have an inconvenient size range and unusual shape distribution that is challenging to study with most standard analytical techniques. They are too large for processes typically used to analyze proteins, such as liquid chromatography (FPLC or HPLC) or electrophoresis. The odd shape distribution of the particles (small spherical vs. elongated filaments that extend from hundreds of nanometers to tens of micrometers long) presents fundamental challenges for analysis/separation by analytical sedimentation on density gradients: virus particle density is independent of size, while filamentous particles have both a greater mass and offer more interactions with the surrounding medium than the spherical particles resulting in opposite forces during sedimentation (i.e., greater mass increases while greater friction decreases the rate of sedimentation). Viruses are too small for light microscopy examination (spherical particles are diffraction limited) and require sample manipulation (purification, fluorescent labeling) for detection that can lead to artefacts. Electron microscopy (negative-strain TEM or cryoEM) is the gold standard, but it is low throughput and laborious, requires extensive sample processing and manipulation that can lead to artefacts, and is highly specialized requiring advanced user training. Related disadvantages (i.e., potential artefacts or laborious processes) apply to available techniques for counting virus particles, such as hemagglutination or TEM experiments. There is currently no good way of separating viruses according to their size for studying the features of one versus another virus particle shape.

What is needed are novel methods for the quantitative measurement of size distributions of pleomorphic virus particles.

### BRIEF SUMMARY

In an aspect, a method of determining a distribution, e.g., size and/or shape, and/or quantification of pleomorphic virus particles in a sample, comprises detecting light scatter off of the pleomorphic virus particles and/or fluorescence of the pleomorphic virus particles, and determining, based on the detecting, the distribution and/or quantification of spherical virus particles, irregular virus particles, and/or filamentous virus particles in the sample.

In another aspect, a method of screening a test drug comprises adding the test drug to a sample comprising pleomorphic virus particles or cells infected with pleomorphic viruses, detecting light scatter off of the pleomorphic virus particles and/or fluorescence of the pleomorphic virus particles, determining, based on the detecting, the distribution and/or quantitation of spherical particles, irregular virus particles, and/or filamentous particles in the sample in the presence of the test drug, and comparing the distribution and/or quantitation in the sample with the test drug to a control distribution and/or quantitation with a control drug or no drug.

A method of treating a viral infection comprises administering to a subject in need thereof an inhibitor of filamentous particle budding or a modulator of virus morphology determined by the method described above, optionally in combination with a cell entry inhibitor, a replication inhibitor, a budding inhibitor, or a combination thereof.

### BRIEF DESCRIPTION OF THE DRAWINGS

FIGS. 1A-C show a model for the role of filamentous particles in adaptation and persistence of viral infections.

FIG. 2 shows a TEM image of sucrose-cushion purified X31HA-Udorn virus particles derived from human Calu-3 cells and a histogram plot of virus-particle length distributions deriving from measurements based on TEM images.

FIG. 3 shows fractionation of Calu-3-derived virus on a sucrose gradient, the TEM images of fractionated virus samples, and histogram plots of virus-particle length distributions deriving from measurements based on several TEM images.

FIG. 4 shows a TEM image of Calu-3-derived virus fractionated by pelleting and a histogram plot of virus-particle length distributions deriving from measurements based on several TEM images.

FIG. 5 shows a flow virometry analysis of Calu-3-derived virus fractionated either on a sucrose gradient or by pelleting and corresponding to virus fractions analyzed by TEM in FIGS. 2 to 4.

FIG. 6 shows a comparison of EM- to flow virometry-derived particle length boundaries.

FIG. 7 shows two methods for particle-yield determination by flow cytometry.

FIG. 8 compares the particle yield determined by flow virometry to particle yield determined by hemagglutination (top) and particle yield determined by two flow-based methods (with or without the reference beads) (bottom).

FIG. 9 shows antibody binding to virus particles causes a corresponding shift in VSSC for the entire virus population including filamentous and spherical particles.

FIG. 10A shows VSSC shift due to antibody binding is the same for all particle types preserving shape profile. The qEV70 columns preserve virus particle shape distributions. Displayed events on all plots were triggered on VSSC-A.

FIG. 10B shows VSSC distribution is the same for viruses detected based on VSSC or fluorescence. A quantum dot-labeled antibody (added to all displayed samples) yields sufficient fluorescent shift to enable detection based on fluorescence of qEV70-purified virus particles and their subsequent shape analysis based on VSSC.

FIGS. 11A-B show a relationship between yield and shape for several virus variants. FIG. 11A is an electron micrograph showing mixed-shape influenza virions. A filamentous and a spherical virion are indicated. FIG. 11B is a plot of virion yield (concentration) versus shape distribution (filamentous virion percent) in the supernatant of cells after a single cycle of infection by WT or mutant influenza viruses under different infection conditions (left), and a plot of virion yield versus median virion size (VSSC) for spherical and filamentous virions for each virus variant and each infection condition (right). FIG. 11C shows a relationship between yield and shape for several virus variants released from MDCK cells in the absence or presence of inhibitors, such as HA-targeting antibodies or the viral M2 or neuraminidase inhibitors. Infecting viruses are either XUd (WT1) or PR8 (WT2) or viruses that exchange one or a pair of segments (WTbaseS1 is WT1 virus with one or two segments from WT2, etc.). Two distinct yield vs. shape trends are observed in different genetic contexts.

FIGS. 12A and B show results for a single infection round of human lung epithelial cell line, Calu3, with A/PR8/34 (12A) or A/Ud/72 (12B) (with X31 or A/Aichi/68 HA) IAV, and using the technology described herein virion yield and shape were quantified. In FIG. 12A, a range of inhibitors of cell entry and/or assembly (antibodies or small molecules) were included either during cell entry (open triangles or circles) or virion assembly (filled triangles or circles). In both cases, a trend where lower virion yields are associated with increased fraction of filamentous particles produced is observed. In FIG. 12B, baloxavir, an inhibitor of IAV mRNA capping, was used. Data for PR8 and XUd viruses are shown on the same plot. Baloxavir was added immediately upon virus attachment to cells and kept over the course of infection. Once again, a trend where lower virion yields are associated with increased fraction of filamentous particles produced is observed. This experiment demonstrates a causal link between reduced virion replication and increased fraction of filamentous particles—shape is phenotypically tunable. This experiment further establishes conditions for shape-inhibitor screen, and conditions for validating inhibitor hits.

The above-described and other features will be appreciated and understood by those skilled in the art from the following detailed description, drawings, and appended claims.

### DETAILED DESCRIPTION

The inventors have developed methods to address how shape affects virus particle function in carefully designed experiments that combined crude virus fractionation with analyses of virus activity on a per-particle basis. Using these methods, the inventors recently demonstrated that filamentous particles resist the effects of neutralizing antibodies or inhibitors of cell entry that inactivate spherical particles. A model has been developed in which filamentous particles enable established infections, such as those by influenza virus, to persist in circulation under changing pressure resulting from immune-system antibodies or man-made inhibitors, or might lead to adaptation to human-cell entry by emerging viruses, such as Ebola virus. The methods and experiments described herein extend this model to show that shape is actively/phenotypically tunable, and thus a more powerful evolutionary strategy than implied by the prevailing models assuming shape distribution is a fixed characteristic specified by a given viral genome. In order to delay and ultimately prevent viral resistance and achieve lasting effects of vaccinations or cell-entry inhibitors and even prevent future pandemics by pleomorphic pathogens, inhibitors of filamentous-particle assembly are urgently needed. The methods described herein can be used to identify such inhibitors.

As used herein, a spherical virus particle is a particle in which all points on the surface are substantially equidistant from the center. Spherical virus particles from the same strain of virus tend to be substantially the same size. A filamentous viral particle, in contrast, has a long dimension and a short dimension from the center, wherein the long dimension is typically at least 1.5- to 2-times longer than the short dimension. Unlike spherical virus particles, whose size is most likely determined by the smallest unit of the packaged genome complement, filamentous virus particles can have a distribution of lengths despite in many cases not incorporating more than a single-genome complement (as in influenza, but some pleomorphic viruses package more than one genome). Virus particles can also be irregular, that is having some asymmetry.

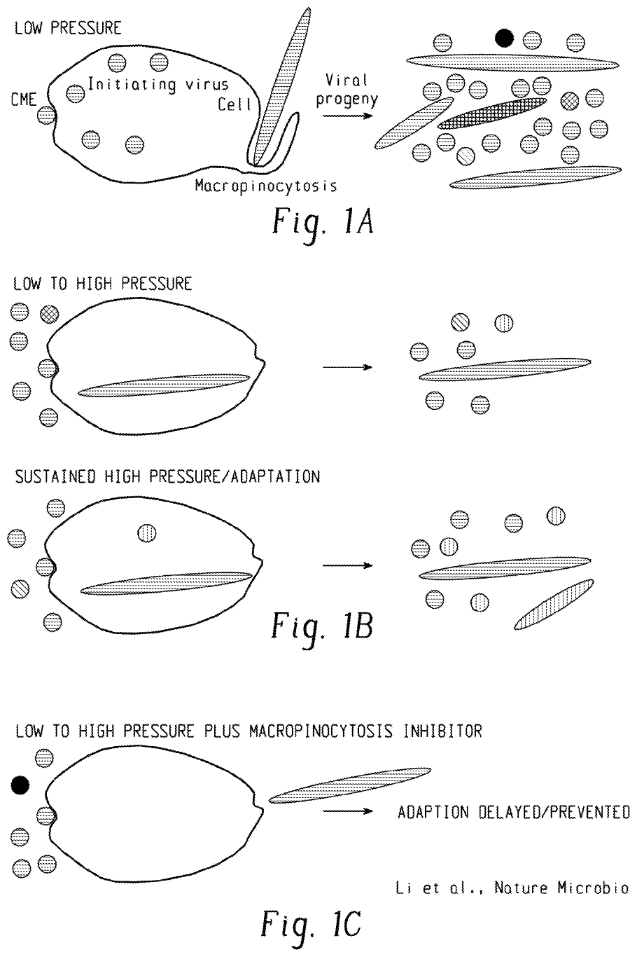

FIGS. 1A-C show a model for the role of filamentous particles in adaptation and persistence of viral infections As shown in FIG. 1, filamentous virions are proposed to have a role in viral adaptation to cell entry pressure. As shown in FIG. 1A, the pleomorphic virus genome encodes for a probabilistic assembly mechanism where virion assembly leads to a distribution of virion shapes. As shown in FIG. 1B, the filamentous particles resist pressure from antibodies or inhibitors that prevent cell entry of spherical particles. Under pressure (e.g., immune system antibodies or a new host), filamentous virions can enter cells and initiate replication that can lead to accumulation of mutations and genetic diversity upon which selection can act and ultimately lead to adaptation (e.g., mutations that prevent antibody binding to virus particles or those enabling efficient entry into a new host). As shown in FIG. 1C, inhibition of filamentous virus assembly or entry, or particle fragmentation can ensure lasting effects of therapies such as immunizations, antibodies and cell-entry inhibitors.

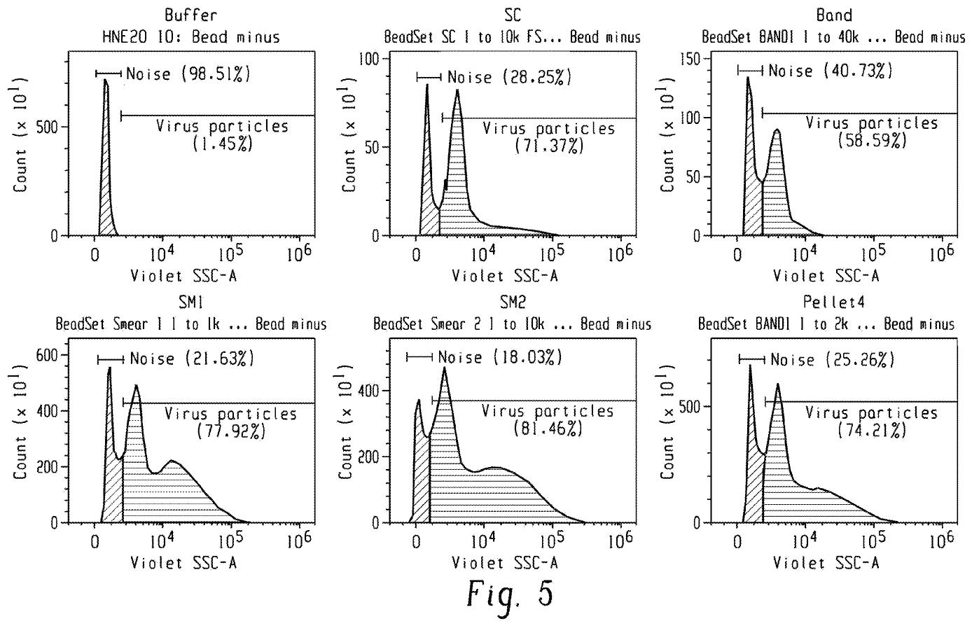

Described herein is a high-throughput, quantitative assay based in fluidics, e.g., flow cytometry, for measuring the distributions, e.g., a size and/or shape, of pleomorphic virus particles (FIG. 5-10). In an aspect, a method of determining a distribution and/or quantification of pleomorphic virus particles in a sample comprises detecting light scatter off of the pleomorphic virus particles and/or fluorescence of the pleomorphic virus particles, and determining, based on the detecting, the distribution and/or quantification of spherical virus particles, irregular virus particles and/or filamentous virus particles in the sample.

In an aspect, the pleomorphic virus comprises an Orthomyxovirus, an Orthopneumovirus, a Paramyxovirus, a Filovirus, or a Coronavirus.

Exemplary pleomorphic viruses include an influenza virus, respiratory syncytial virus, measles virus, Ebola virus, Nipah virus, Marburg virus, severe acute respiratory syndrome coronavirus (SARS-COV), Middle East respiratory coronavirus (MERS-COV), SARS-COV-2 and Hendra virus.

In an aspect, detecting comprises near UV detection (180-400 nm), 405 nm laser light violet light side scatter (VSSC), detecting a fluorescent label on the virus particles, or a combination thereof. In an aspect, detecting is done using a system that provides fluidics of a sample containing virus particles and interrogation of the particles with detection of light scatter off of the pleomorphic virus particles and/or fluorescence of the pleomorphic virus particles. Exemplary methods include flow virometry and other microfluidics methods such as the use of microfluidic chips including a pattern of microchannels permitting virus-containing buffer flow and interrogation with light and including detection of scattered light (ultraviolet to violet range) or fluorescence emission.

In an aspect, the distribution and/or quantification of pleomorphic virus particles is determined using 405 nm laser light (or similar, i.e., in the UV-Violet range), i.e., violet light side scatter (VSSC). Advantageously, in the VSSC approach, a sufficiently short wavelength of light is used so that the light can be scattered off of the virus particles. It is this scatter that is measured. In this method, a sensitive detector can be used to facilitate detection.

In addition to or as an alternative to the VSSC approach, detecting can comprise detecting a fluorescent label on the virus particles. For example, the virus particles can incorporate a fluorescent protein, the virus particles (viral membrane or protein/nucleic acid components) can be fluorescently labeled, the virus particles can be bound by a fluorescent molecule, and the like. Exemplary fluorescent proteins include green fluorescent protein, yellow fluorescent protein, red fluorescent protein, turquoise fluorescent protein, and the like. Exemplary fluorescent labels include fluorescein, rhodamine, isothiocyanates (TRITC, FITC), Texas Red, Cy2, Cy3, Cy5, APC, AlexaFluor® (Invitrogen, Carlsbad, Calif.) range of fluorophores, JaneliaFluor® dyes (Tocris Bioscience), Atto dyes, Quantum-dot labeled probes or antibodies, and the like. Exemplary membrane dyes include Octadecyl Rhodamine B (R18), long-chain carbocyanines such as Dil, DiO, DiD, DiR, and the like.

Advantageously, the methods described herein have excellent sensitivity. In an aspect, the sensitivity is at least 10⁷ virus particles/ml. Even greater sensitivities may be achieved with purified samples.

Exemplary samples include supernatants from an infected cell, such as a cell from a patient, infected animal, or a cultured cell. Exemplary cultured cells include Madin-Darby Canine Kidney (MDCK) cells, chicken embryo kidney cells (CEK), chicken embryo fibroblast cells (CEF), quail fibroblast cells (QT6), chicken hepatocellular carcinoma cells (LMH), primary rhesus monkey kidney cells (RhMK), primary rabbit kidney cells, human embryonic kidney (HEK) 293T cells, MRC-5, human foreskin fibroblasts, Hep-2, human alveolar epithelial cells (A549), cultured human airway epithelial cells (Calu-3), and the like.

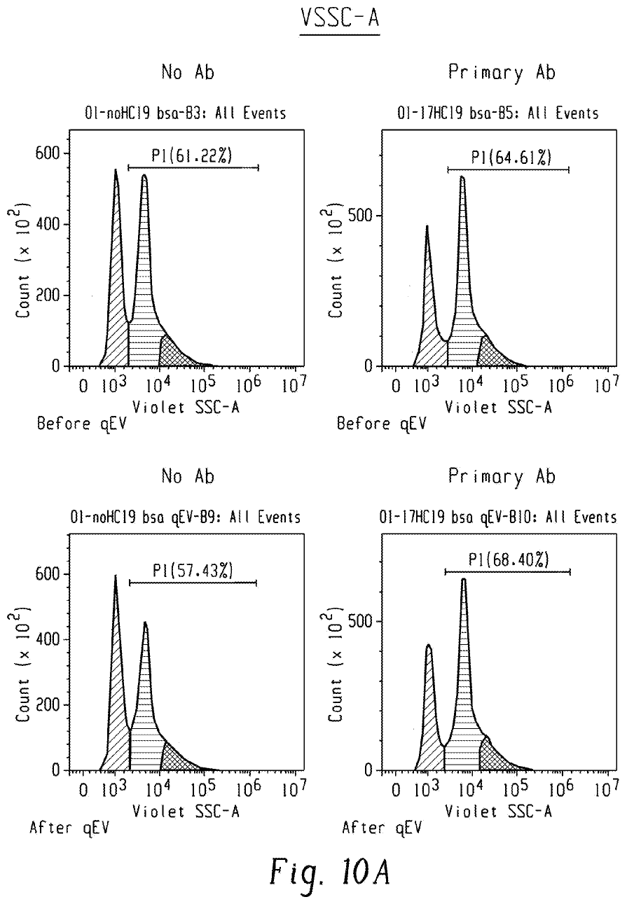

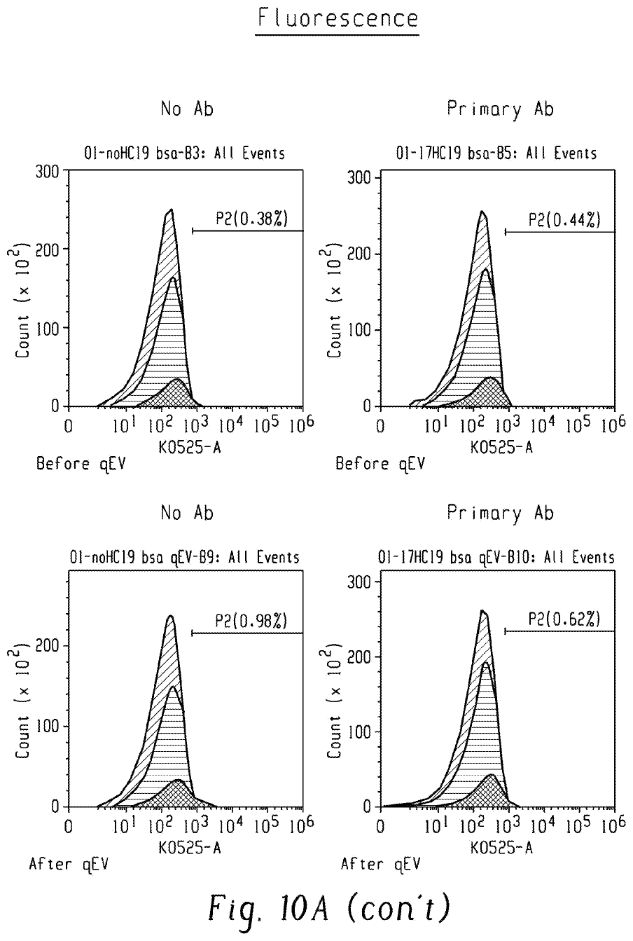

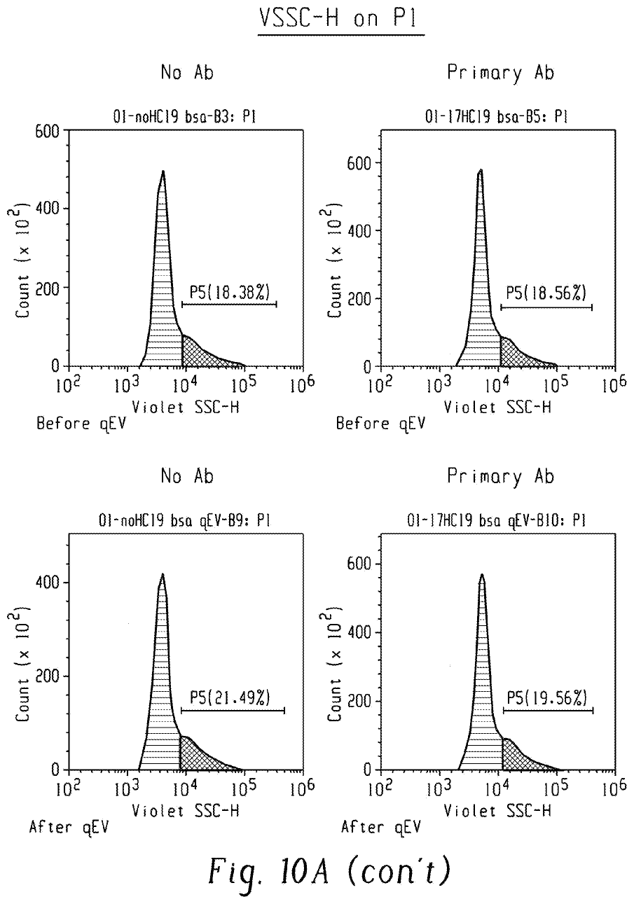

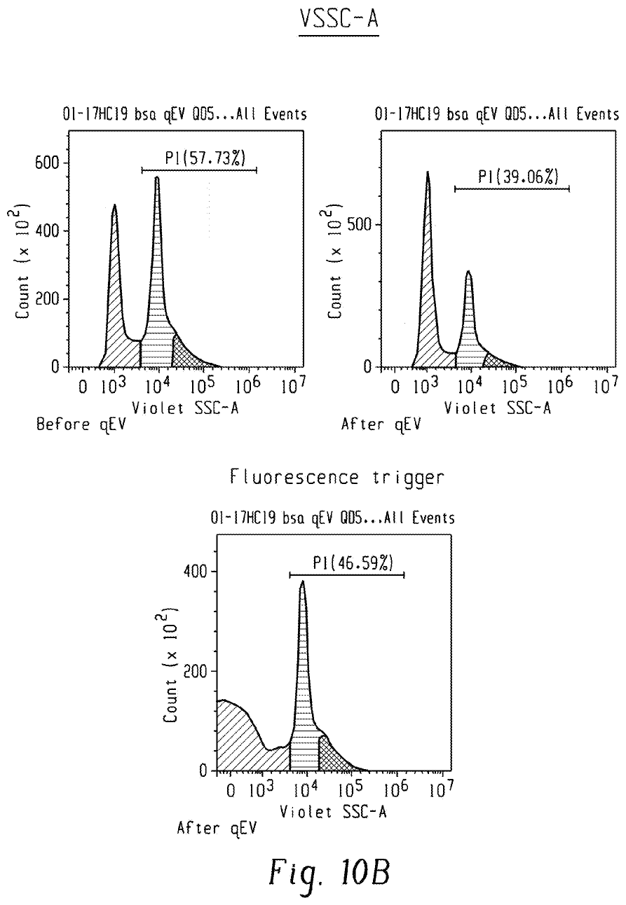

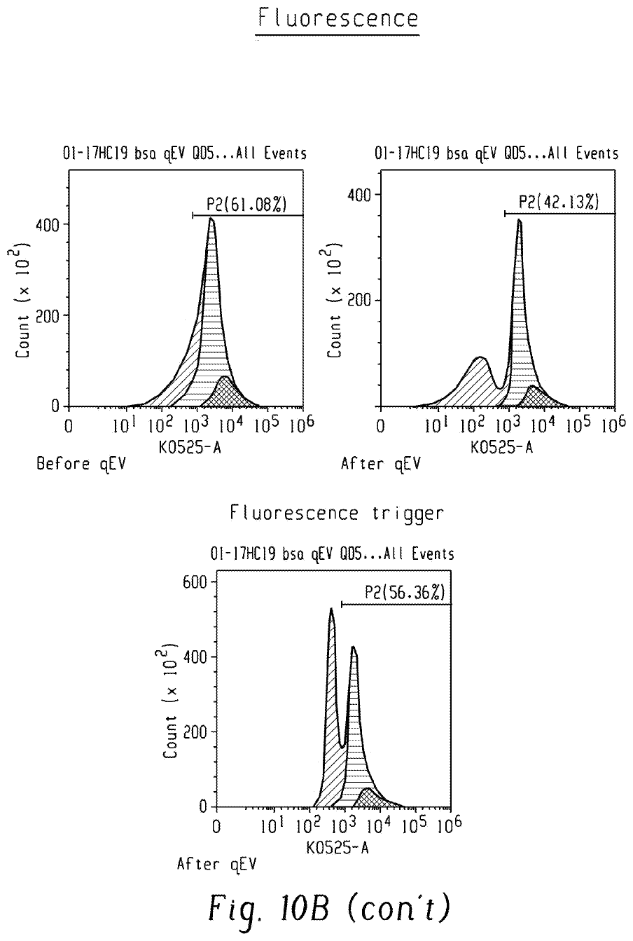

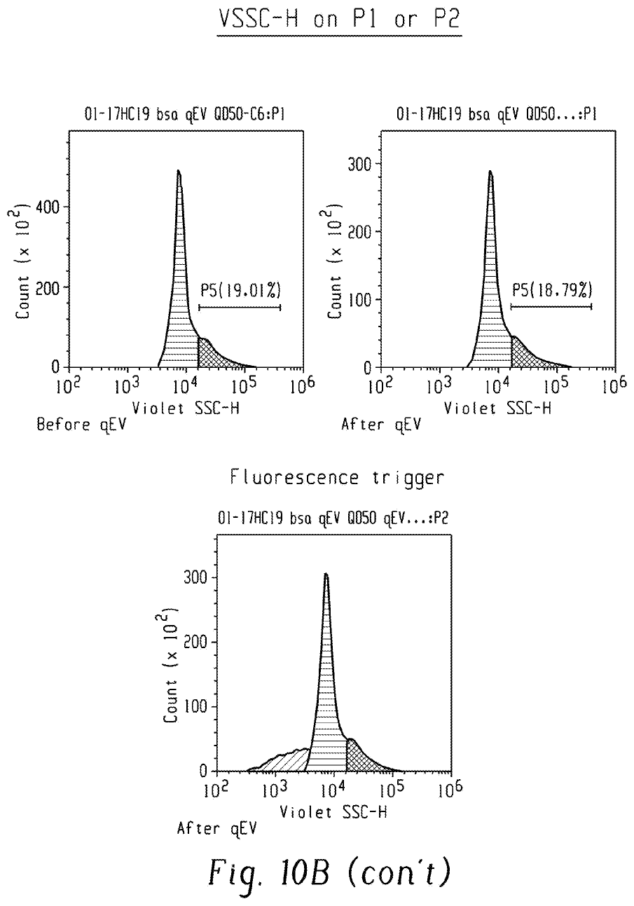

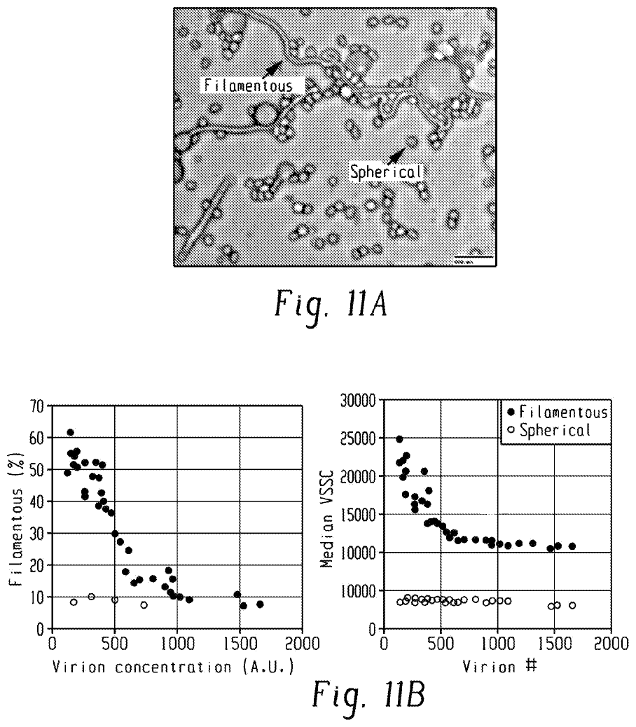

In an aspect “dirty” or unpurified samples (from cellular extracts, infected animals, or human patients) can be treated with fluorescent antibodies, and VSSC can be measured from the fluorescent samples to obtain shape/size distribution of virus particles within these samples. See FIG. 10.

In an aspect, the sample is undiluted or treated only by dilution prior to detecting. Advantageously, the methods described herein can be performed using only a simple dilution of the sample in a buffer and no other manipulation, such as sample concentration, purification, or labeling is required. Exemplary buffers include phosphate, HEPES, Tris, PIPES, MES, citrate, and the like.

Alternatively, the sample may be purified, such as by density gradient ultracentrifugation, filtration, nanofiltration, precipitation, chromatography such as size-exclusion chromatography, antibody-conjugated magnetic beads, and the like.

In an aspect, the assay is a high throughput assay such as a 96-well format assay. High throughput assays will enable library screens for inhibitors of filamentous-particle assembly, for example. The method can be carried out in conventional 96-well microtiter plates, or in any other container or on any surface capable of holding liquid samples and of being scanned by flow virometry. Examples include 12-well, 24-well, 384-well, 864-well plates, and microscope-slides.

The high throughput (automated, up to a 96-well plate format), high sensitivity (requires low particle concentrations), and quantitative (derives particle-size distributions and counts particles) assays described herein avoid processing artefacts, i.e., they do not require any specimen manipulation such as purification or labeling that could alter specimen properties, although purification and/or labeling may be employed. By including antibodies as described above, the approach can be extended to measure particle composition (relative incorporation of viral surface proteins), antigenicity (including measurements of antibody affinity for virus particles), the antibody mode of action (e.g., simple binding vs. particle crosslinking), as well as determining these properties and virus particle shape/size distribution from animal or patient samples.

In an aspect, the method further comprises adding a fixed amount of fluorescent beads to the sample prior to detecting, and obtaining a ratio of total virus particles, spherical virus particles and/or filamentous virus particles to beads. In this aspect, the number of total virus particles, spherical virus particles and/or filamentous virus particles can be quantified as a function of the fraction of bead particles for a constant input bead amount. This method yields arbitrary units unless the actual bead concentration is known (or our alternative method is employed, see below) and can be used for comparing relative virus yields for different infections, virus preparations, or purification fractions or intermediates. This approach has been validated by comparing the results to values obtained by a standard hemagglutination assay for a series of purification or fractionation intermediates of a given virus preparation. Advantageously, this method can be employed across pleomorphic virus types, strains, and mutants to yield relative virus particle concentrations that can be compared across virus types, strains, and mutants (HA results fail to yield these cross-type comparisons for even closely related viruses if they differ in their affinity for receptors on red blood cells).

Exemplary fluorescent beads include 170 nm-in-diameter FluoSpheres™ (Invitrogen). Fluorescence on the beads may be used as a way to differentiate input beads from virus particles in the case that the light-scatter profile of beads overlaps with that of virus particles. If beads of known concentration are used, they can allow the user to determine absolute virus particle (total, spherical, and/or filamentous) counts by the above method.

In an aspect, when a defined flow rate is used, the method can comprise determining the absolute concentrations of the total virus particles, the spherical virus particles and/or the filamentous virus particles in the sample. When a defined flow rate is used, a given time interval corresponds to a precise volume. By defined flow rate it is meant using the same flow rate from sample to sample so that the same ‘gate’ on a time plot corresponds to the same volume. When the flow rate is defined (i.e., calibrated), the count of virus particles that pass in front of the detector in a given time interval yields absolute virus particle concentration, i.e., the number of virus particles per volume of buffer. Multiplying that value with the dilution factor for the given sample, provides the original concentration of virus particles. Advantageously, this method can be employed across pleomorphic virus types, strains, mutants to yield absolute virus particle concentrations that can be compared across virus types, strains, mutants.

In yet another aspect, the method further comprises adding an antibody specific for a viral surface protein to the sample, wherein a shift in the VSSC is proportional to an amount of the viral surface protein in the virions at saturating antibody concentrations. In an aspect, the antibody comprises a fluorescent label, or wherein the method further comprises contacting the sample with a fluorescent secondary antibody that binds the antibody specific for viral surface proteins. Exemplary viral surface proteins include hemagglutinin, neuraminidase, proton channel M2 for influenza viruses, or surface proteins specific for any virus of interest such the GP for Ebola.

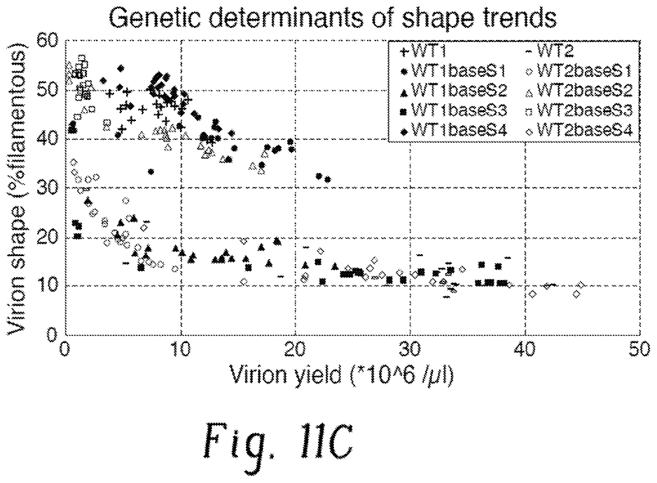

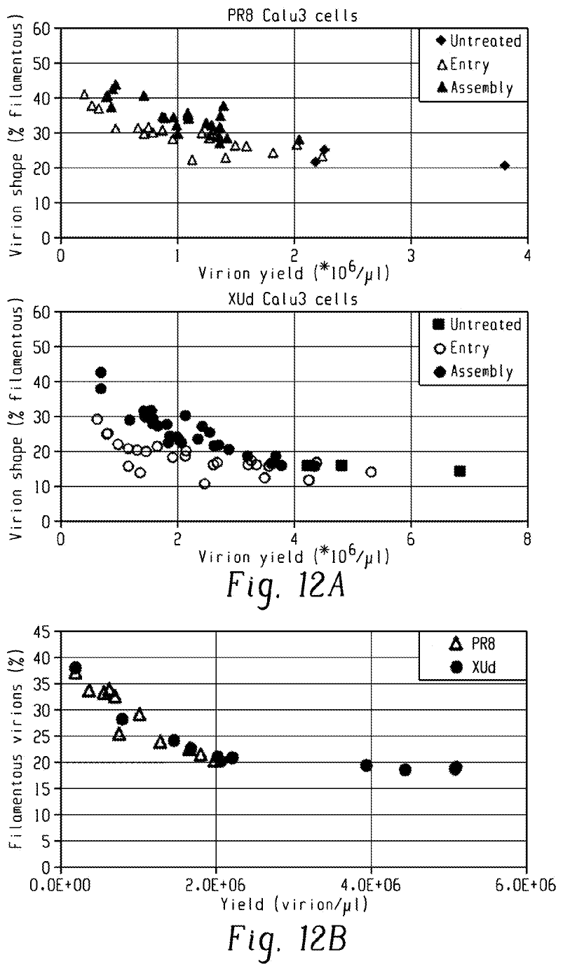

In a further aspect, the method further comprises adding a test drug to the sample and determining the effect of the test drug on the spherical virus particles, irregular virus particles, and/or the filamentous virus particles in the sample. The shape/yield relationship for viruses produced in the presence of the drug revealed by the experiments in FIGS. 11 and 12 is key in determining whether a drug fundamentally affects virion shape. In the assay, the drug prevents filamentous shape/large virions even when virion output/yield is low (due to this or a different drug/treatment). The identification of inhibitors of filamentous particle assembly and optionally combining these identified inhibitors with cell-entry inhibitors or vaccinations could delay or prevent viral adaptation and thus enable eradication of viruses from circulation in humans, or, in the case of emerging pathogens, prevent new viral outbreaks/pandemics. Also, the identification of treatments modulating particle shape and/or composition and the discovery of novel viral or cellular drug targets involved in virus assembly, budding and release are possible with the methods described herein.

In an aspect, a method of screening a test drug, comprises adding a test drug to a sample comprising pleomorphic virus particles or cells infected with pleomorphic viruses, detecting the pleomorphic virus particles, determining, based on the detecting, the distribution and/or quantitation of spherical particles and/or filamentous particles in the sample in the presence of the test drug, and comparing the distribution and/or quantitation in the sample with the test drug to a control distribution and/or quantitation with a control drug or no drug. The determining can include 405 nm (UV-Violet range) laser light scatter, i.e., violet light side scatter (VSSC), detecting a fluorescent label on the virus particles, or a combination thereof as described above.

The methods described herein provide for probing the activity of various antibody and small-molecule treatments in modulating virus budding and particle shape and enables unbiased screens for inhibitors of filamentous particle budding and modulators of virus morphology. The methods further provide screens for compounds that might modulate the shape of (i.e., fragment) already assembled particles. The utility extends to all circulating and emerging pleomorphic viral pathogens including but not limited to influenza, respiratory syncytial virus, measles, Ebola, Nipah, and Hendra viruses.

The test drug can be an antibody or a small molecule drug. In an aspect, the test drug is an inhibitor of filamentous particle budding/assembly or a modulator of virus morphology. Advantageously, the test drugs can prevent filamentous virion assembly under conditions of reduced virus replication (e.g., immune pressure, drug pressure, or suboptimal entry into cells of a new host).

Exemplary control drugs include reported inhibitors of virus cell entry (anti-HA antibodies such as HC19 IgG, Medi8852 IgG, Sa 48-1A6-6 IgG), viral mRNA capping and replication (baloxavir marboxil), internal virion acidification (rimantadine), virus budding (anti-HA antibodies such as HC19 IgG, Medi8852 IgG, Sa 48-1A6-6 IgG, anti-M2 antibodies 14C2 and mAb65), virion release (oseltamivir). These drugs can serve as negative controls because they modify virus morphology in accord with their effect on virus yield (for increased virus production, they reduce the fraction of filamentous particles, and for decreased virus production, they increase the fraction of filamentous particles). At higher concentrations oseltamivir is an exception as it reduces virion yield, induces virion aggregation, but the size of individual virus particles remains enriched in spherical particles. In addition, these known inhibitors (except NA inhibitors such as oseltamivir) will serve as reference conditions that facilitate filamentous virion production in whose presence we will screen for prevention of virion release by test compounds. A desired compound will decrease the fraction of filamentous particles and decrease virus production or decrease the fraction of filamentous particles without an effect on virus production (in the latter case, it would be combined with inhibitors of virus replication (examples listed above) and serve to delay/inhibit resistance to said inhibitors).

In an aspect, the test drug is a member of a library of compounds. Advantageously, high throughput screening methods of a combinatorial chemical or antibody library containing a large number of potential therapeutic compounds can be done using the methods described herein. Such “combinatorial chemical libraries” are then screened to identify those library members (particular chemical species or subclasses) that display a desired characteristic activity, e.g., a reduction in virion yield and reduction of filamentous virion fraction in the virus population, or reduction of filamentous virion production when virion yield is reduced by other control treatments. The compounds thus identified can serve as conventional “lead compounds” or can themselves be used as potential or actual therapeutics.

In an aspect, a combinatorial library is a library in which one or more functional groups of the lead compound are varied, e.g., by derivatization. Thus, the combinatorial library can include a class of compounds which have a common structural feature (e.g., framework). A combinatorial chemical library is a collection of diverse chemical compounds.

In an aspect, the method of screening a test drug further comprises determining a number of total virus particles, a number of spherical particles and/or a number of filamentous particles or their relative numbers (fraction of either type in the population) in the presence and absence of the test compound. This method can include using antibodies for specific viral surface proteins as described above.

In yet another aspect, the method of screening a test drug further comprises determining the effect of the test compound on virus assembly, virus budding, and/or virus particle release. Advantageously, the methods described herein allow one to study shape-yield trends in pleomorphic virus particles which can allow the identification of true modulators of virus morphology (i.e., compounds that change the shape-yield relationship and/or prevent filamentous particle production when the overall virus production is reduced).

In an advantageous method, the pleomorphic virus particles are either from a mutant virus or viral strain that yields a large fraction of pleomorphic viruses under normal (no inhibitor) conditions (screen strain/mutant) or from a WT virus grown under the conditions that favor filamentous virion production (under inhibitor pressure, suboptimal temperature, or with cell targets with lower permissiveness). High throughput screening methods as described herein can assist in the identification of inhibitors that modulate shape trends. Compound/inhibitor hits will be validated under a full range of replication conditions of WT viruses to obtain yield vs. shape curves and compared to such curves in the absence of hit compounds (see FIG. 12).

Enabling quantitative analyses of virus particle shape and composition are key breakthroughs of the present invention. Concurrent counting of virus particles adds yet another key dimension to the same measurements. Pleomorphic virus particles have an inconvenient size range and unusual shape distribution that falls through the cracks of most standard analytical techniques. They are too large for processes typically used to analyze proteins, such as liquid chromatography (FPLC or HPLC) or electrophoresis. Their odd shape distribution (small spherical vs. elongated filaments that extend from hundreds of nanometers to tens of micrometers long) presents fundamental challenges for analysis/separation by analytical sedimentation on density gradients: virus particle density is independent of size, while filamentous particles have both a greater mass and offer more interactions with the surrounding medium than the spherical particles resulting in opposite forces during sedimentation (i.e., greater mass increases while greater friction decreases the rate of sedimentation). Viruses are too small for light microscopy examination (spherical particles are diffraction limited) and require sample manipulation (purification, fluorescent labeling) for detection that can lead to artefacts. Electron microscopy (negative-strain TEM or cryoEM) is the gold standard, but it is low throughput and laborious, requires extensive sample processing and manipulation that can lead to artefacts, and is highly specialized requiring advanced user training. Related disadvantages (i.e., potential artefacts or laborious processes) apply to available techniques for counting virus particles, such as hemagglutination or TEM experiments. The methods described herein overcome all of the limitations of the above listed technologies. The methods described herein enable high-throughput, automated counting of particles and quantification of the relative fraction of spherical vs. filamentous particles in a population. It is very sensitive requiring only approximately 0.5-2 μl of unprocessed/unmanipulated infectious cell supernatant avoiding processing artefacts and enabling scale-up needed for inhibitor screens that test many compounds for their effects on particle size/shape and/or numbers in parallel. The screens can be performed in cell culture (screens for cellular/viral modulators of budding or assembly) or on purified viruses (screens for compounds that alter particle shape of assembled virions).

In an aspect, a method of treating a viral infection comprises administering to a subject in need thereof an inhibitor of filamentous particle budding or a modulator of virus morphology determined by the method described above, optionally in combination with a viral cell entry inhibitor. The inhibitor of filamentous particle budding or modulator of virus morphology can be administered simultaneously or sequentially with a replication inhibitor (such as inhibitor of cell entry). Such coadministration is predicted to delay viral resistance.

Exemplary inhibitors of viral cell entry include neutralizing antibodies targeting cell-entry glycoproteins including broadly neutralizing antibodies and small-molecule inhibitors of cell entry such as the broad antiviral inhibitor Arbidol.

The invention is further illustrated by the following non-limiting examples.

### EXAMPLES

### Methods

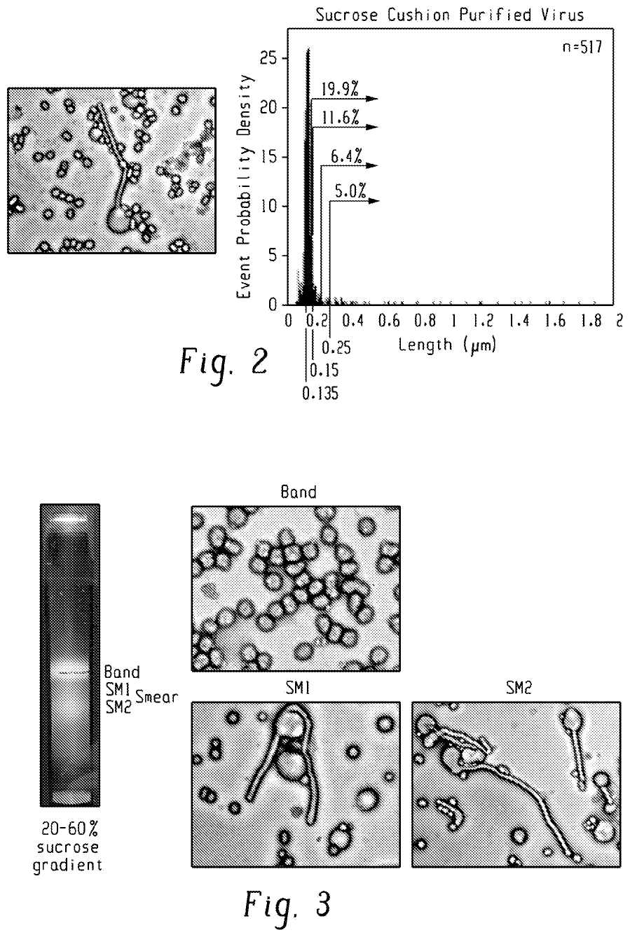

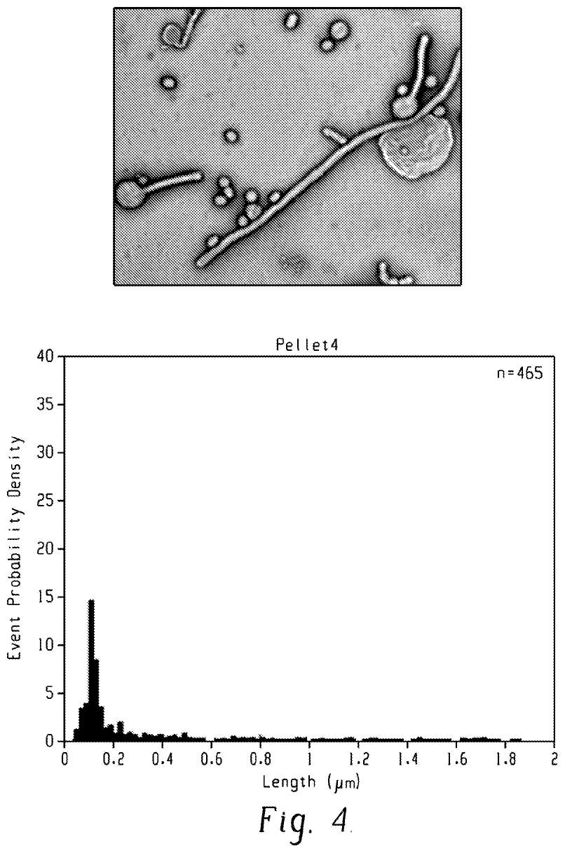

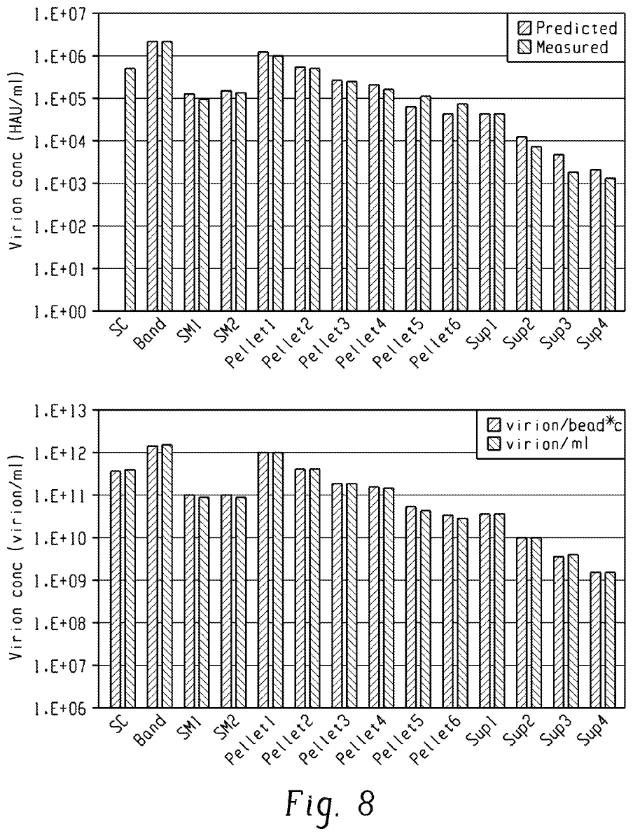

Media and buffers: Purified-virus storage buffer HNE20 includes 20 mM HEPES NaOH pH 7.4, 150 mM NaCl and 0.2 mM EDTA. All infections were performed in OptiMEM™ media (Thermo Fisher Scientific), either in the presence or absence of TPCK-trypsin (Sigma) at 1 μg/ml to activate HA on released virions.
Virus strains: X31HA/Udorn (H3N2) has A/Aichi/68 (X31) HA and the remainder of the segments from A/Udorn/72 strain (described in Ivanovic et al, “Kinetics of Proton Transport into Influenza Virions by the Viral M2 Channel”, PLOS One 7(3): e31566, 2012). This is the WT virus used in FIGS. 1 to 10. FIG. 11B additionally used A/PR/8/34 (H1N1) influenza (PR8), X31HANA/Udorn with both HA and NA from A/Aichi/68, and each X31HA/Udorn and X31HANA/Udorn with D188N mutation in HA1. FIG. 11C additionally used PR8 influenza that exchanges HA or M genome segments, M2 gene, or HA and M genome segments together with the corresponding genome segments or genes from XUd, and XUd influenza that exchanges the same set of genome segments or genes with those from PR8. The virus in FIG. 12A is PR8 influenza and the remaining FIG. 12 panels include both PR8 and XUd influenza.
Virus purification: Human lung cancer Calu-3 cells were infected at a multiplicity of 12 PFU (plaque forming units)/ml, and supernatants were collected 45-hrs post infection. Virus-containing supernatants were passed through a 20% (w/v) sucrose cushion in HNE 20 to purify and concentrate viruses (100,000×g for 2.5 hrs), and then either fractionated on a 20-60% (w/v) sucrose gradient (as in Ivanovic et al., PLOS One, 2012) or subjected to 1 to 6 cycles of centrifugation at 3250 rcf for 1.5 hrs at 4° C. (as in Li et al., “The shape of Pleomorphic Virions Determines resistance to Cell-entry Pressure”, Nature Microbiology, 6, pp. 617-629, 2021). For each centrifugation step, virus was diluted to 900 μl in HNE 20. After each spin, 850 ul of the supernatant volume was removed, and the pellet was resuspended in HNE 20 over 6-14 hours at 4° C. The final spin constituted the PELLETn fraction where n indicates the number of spins performed. Supernatants were combined and concentrated by a 1-hr spin at 100,000×g (the SUP refers to the combined supernatants after the final spin, and SUPn indicates the supernatant of an n-th spin). Virus purification away from unbound antibodies (FIG. 10) was achieved by a passage through a qEV70 column according to manufacturer recommendations (Izon).
Electron Microscopy: The procedure was previously described (Li et al., Nature Microbiology 2021). Briefly virus concentrations were adjusted to between 2*10⁴ and 1*10⁵ HAU/ml (HAU refers to standard hemagglutination units derived using chicken red blood cells). From the current measurements in FIG. 8, those HAU/ml values correspond to virus particles concentrations of 2*10¹⁰-1*10¹¹ virions/ml (see next methods section, Determination of Particle Concentrations). Viruses were stained with 2% phosphotungstic acid. Virus lengths were determined using custom MATLAB codes as described (Ivanovic et al., PLOS One 2012). 25 to 50 images were processed to yield the distributions shown in FIGS. 2 and 4.
Determination of particle concentrations: Purified virus fractions were diluted to approximately 30-100 HAU/ml in HNE 20 and analyzed by flow virometry on the CytoFLEX S platform and results were analyzed using CytExpert software version 2.4.0.28 (Beckman Coulter Life Sciences). Virus dilutions were run at the constant flow rate of 10 μl/min and the results were recorded after 2 minutes of flow until 500,000 total events were recorded within the Particle gate (FIG. 7, left panel). VSSC voltage was adjusted to 300. VSSC was used as the event trigger with manually adjusted threshold of 1400 in VSSC Height. 170 nm FluoSpheres™ (Invitrogen) (excitation/emission maxima of 505/515) were used as a constant reference bead at 1:300 dilution.
Bead method for determining particle concentrations (yields arbitrary units): Particle concentration in purified virion fractions in reference to beads was calculated as % virions/% beads*virus dilution where bead concentration was kept constant in each sample (1:300 dilution) (FIG. 7, middle panel). To obtain values for the actual virion concentration (yields virion/ml), the total number of particles in the virion gate (FIG. 7, middle panel) within a defined time interval (FIG. 7, right panel) was multiplied by the input virus dilution and divided by the total volume flown during this time interval (volume=flow rate*time (min within the interval)=10 μl/min*0.267 min=2.67 μl=0.00267 ml).

To generate yield shape curves the following infection scheme was followed. Cells were infected at multiplicity of 3 PFU/cell for 24 hrs in the absence of trypsin to ensure a single-cycle of infection. 35 μl of virus was added to confluent monolayers of cells in 24-well plates. Virus was allowed to attach for 1 hr at room temperature. The unattached virus was washed away with two HBSS washes, and 300 μl OptiMEM media was added to wells. The following inhibitors were used to generate yield vs. shape curves (FIG. 11C) at indicated concentration range: anti-HA antibodies HC19 IgG (0.6-20 nM), Medi8852 IgG (17-536 nM), Sa 48-1A6-6 IgG (0.3-10 nM), an M2 inhibitor rimantadine (0.8-200 μM), and a neuraminidase inhibitor Oseltamivir (1.2-99 nM). The inhibitors were either added 4 hrs post infection. The following inhibitors were used to generate yield vs. shape curves (FIG. 12) at indicated concentration range: anti-HA antibodies HC19 IgG (0.6-20 nM), Medi8852 IgG (17-536 nM), Sa 48-1A6-6 IgG (0.3-10 nM), internal virion acidification inhibitor rimantadine (1-99 μM), and anti-M2 antibody 14C2 (12-200 μg/ml), and viral mRNA capping and replication inhibitor baloxavir marboxil (0.1-11 nM). Baloxavir was added immediately after virus attachment in the infection optimum media and kept in the infected cell supernatant during the infection. The rest of the inhibitors were either added 4 hrs post infection (assembly on FIG. 12B\A) or were used to preincubate viruses, then were present during both virus attachment (1 hr at room temp) and the first 4 hrs of infection at 34 C (entry on FIG. 12A) at which point media was changed to OptiMEM without inhibitors. Virus supernatants were diluted in HNE20 as described above and analyzed by flow cytometry to determine virus particle shape/size distributions and yields.

### Example 1: Enrichment for Spherical and Filamentous Virions

An enrichment strategy for spherical and filamentous virions uses cycles of low-force sedimentation, and the analysis of virion size distributions using negative-stain TEM. 25 to 50 such images are taken, and diameters or lengths of particles were measured using custom codes that involve manual clicking along the length of particles. The number of particles that went into each plot is indicated on the plots. For quantitative determination of virion size, electron microscopy (negative-strain TEM or cryoEM) is the gold standard.

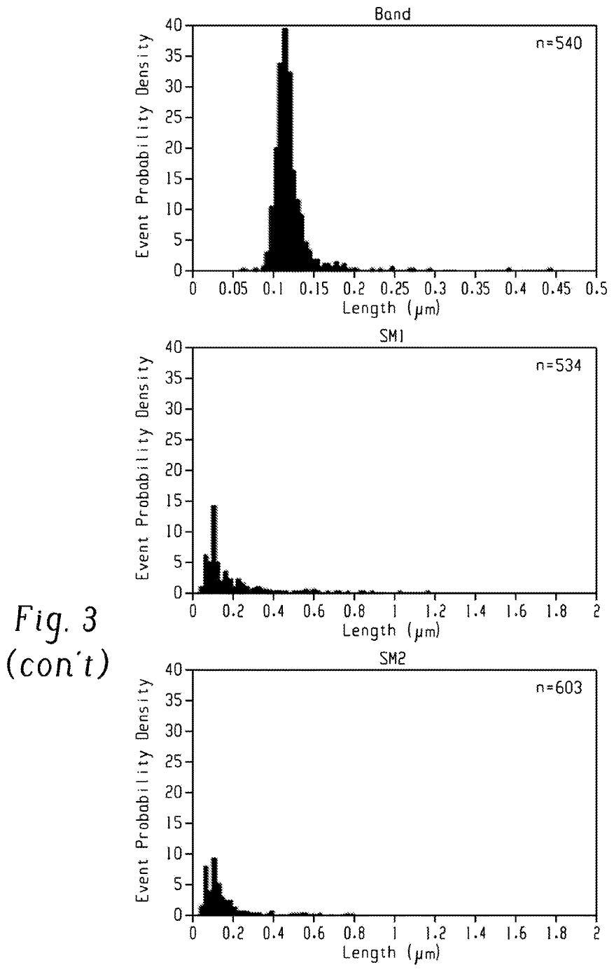

The influenza virus (X13HA/Udorn strain) was grown on Calu-3 cells, purified through a sucrose cushion, and the resulting pellet was visualized by TEM after phosphotungstic acid (PTA) negative staining. As shown in FIG. 2, the majority of particles are spherical for this virus on this cell line, but a small fraction (approximately 5%) of filaments of various lengths are also present. As shown in FIG. 3, half of the sucrose-cushion purified virus was further fractionated on a sucrose gradient (rate zonal centrifugation), and 3 fractions were collected as indicated on the gradient—Band, Smear 1 (SM1) and Smear 2 (SM2). For smears, the fuzzy region of the gradient below the main band was collected as two equal parts. Representative PTA-stained TEM images for these fractions are shown on the right. The Smear fractions are enriched in filamentous particles compared to the sucrose cushion virus. As shown in FIG. 4, 25 to 50 such EM images were taken and particle diameters (for spherical) or lengths (for filamentous) were measured as indicated above. The resultant diameter/length distributions are shown on the bottom. The number of particles that went into each plot is indicated in Figures. The other half of the sucrose-cushion purified virus was subjected to pelleting fractionation, and the results for the 4^{th} pellet are shown in FIG. 4. Because negative-strain TEM and cryoEM are low throughput and laborious, require extensive sample processing and manipulation that can lead to artefacts, and are highly specialized requiring advanced user training, the inventor developed an alternative strategy to identify and quantify spherical and filamentous virions. The EM-derived quantification in FIGS. 2-4 was used as a means of validating the novel approach.

### Example 2: Analysis of Spherical and Filamentous Virion-Containing Fractions by Flow Virometry

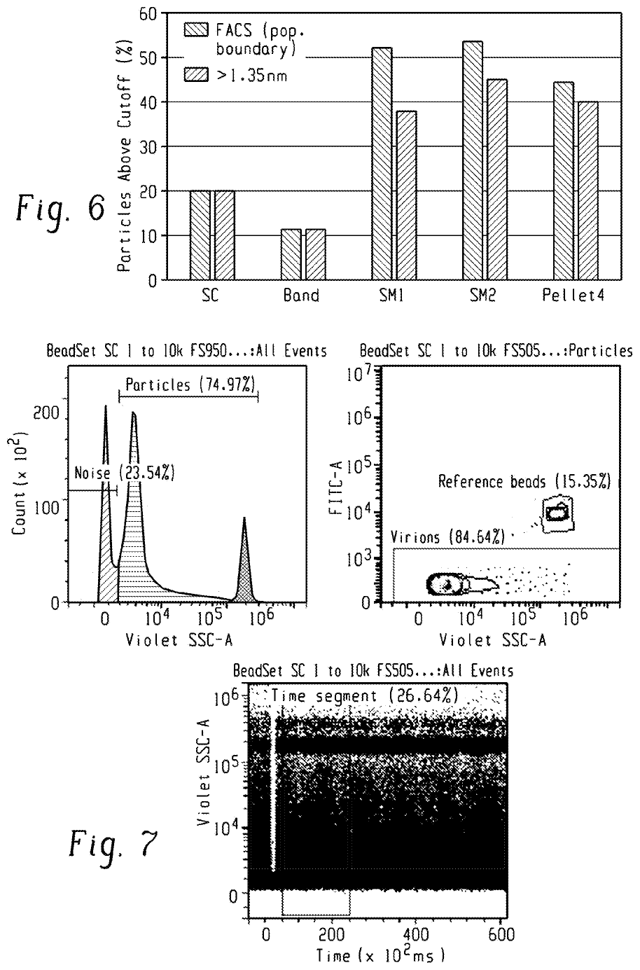

An assay based in flow virometry for measuring the size distributions of pleomorphic virus particles was developed. The basic principle of the assay is that larger particles or longer filamentous particles cause greater scattering of the 405 nm laser light (Violet Side Scatter, or VSSC) than the spherical particles. As shown in FIG. 5 for various sucrose gradient fractions, the amount of scatter of the 405 nm laser is proportional to particle size. Either a tail or a distinct filamentous particle hump is noticeable at higher VSSC values depending on the extent of filamentous particles in each prep, and the scatter-derived distributions resemble the EM-derived distributions but many more particles (500,000 vs. approximately 500) can be analyzed in a matter of minutes. FIG. 6 shows a comparison of the EM and flow based filamentous virion quantification. In FIG. 6, the percent of virions in the second hump were determined and compared to the % of virions longer than 135 nm as measured from the TEM images. There is a close correlation between measured values for the two approaches (EM and flow virometry). It was concluded that the particle length/diameter distribution in the first virion hump is approximately 55 to 135 nm, a value closely matching the previous measurements for the spherical influenza particle length/diameter distributions derived from EM (Ivanovic et al., “Influenza-virus membrane fusion by cooperative fold-back of stochastically induced hemagglutinin intermediates, eLife, 2012, e00333). These results establish the boundary between the spherical and filamentous viruses in VSSC profiles. The combined results further validate the use of the flow virometry-based method for establishing virion size distributions.

### Example 3: Method of Quantifying Virion Numbers

A fixed dilution of fluorescent beads can be added to the sample to provide the ratio of virions to beads. A standard method for quantifying a correlate of virion numbers is the hemagglutination assay. The HAU/ml value is proportional to virion numbers in a given preparation of virus and is related to the avidity of virus particles for receptors on red blood cells (in turn determined by HA affinity for the sialic-acid receptor). This is a convenient assay for comparing virus concentrations among the same viruses or viruses with similar HA-receptor affinity. The assay can be used to validate the novel method described herein where different fractions of the same virus prep are compared (so same HA, same HA affinity for the sialic acid), but this point also illustrates a major limitation of the standard hemagglutination assay (cannot be used to compare particle yields for HA mutants, or distinct influenza strains, different virus types, etc.). The assay described herein overcomes this limitation and potential applications include vaccine studies (e.g., evaluation), vaccine development, drug studies (evaluation), drug development, and the like. The hemagglutination units (HAU) was determined for the sucrose-cushion purified virus and we measured particle/bead ratio for a number of different purified fractions (see Virus Purification under Methods above). From the values for HAU/ml and particle/bead for the sucrose cushion virus, a conversion factor was obtained (8.7) for predicting HAU/ml for the remaining fractions as follows. Predicted HAU/ml=particle/bead*8.7. HAU/ml was then measured for the remaining fractions. As shown in FIG. 8, there was a close correlation between the predicted and measured values validating the approach for determining physical particle equivalents. This approach does not depend on any physical qualities of the virus (e.g., receptor affinity, etc.) other than particle size (but virions of 55 nm in diameter and greater can be detected) so it can be used for any influenza virus strain and many other viruses. This approach has been extended to enable determination of actual virion/ml value for the various fractions (see Methods above). FIG. 8, bottom validates it and establishes a conversion factor of 7.1*10⁶ (constant) by which to multiply particle/bead values to obtain particle (virion)/ml. Either method works, the latter is even simpler, gives actual virion/ml, and the two sets of results perfectly agree with each other. Furthermore, this experiment establishes the value for virion/HAU for this virus strain as 10⁶.

### Example 4: Use of Antibodies That Bind Viral Surface Proteins

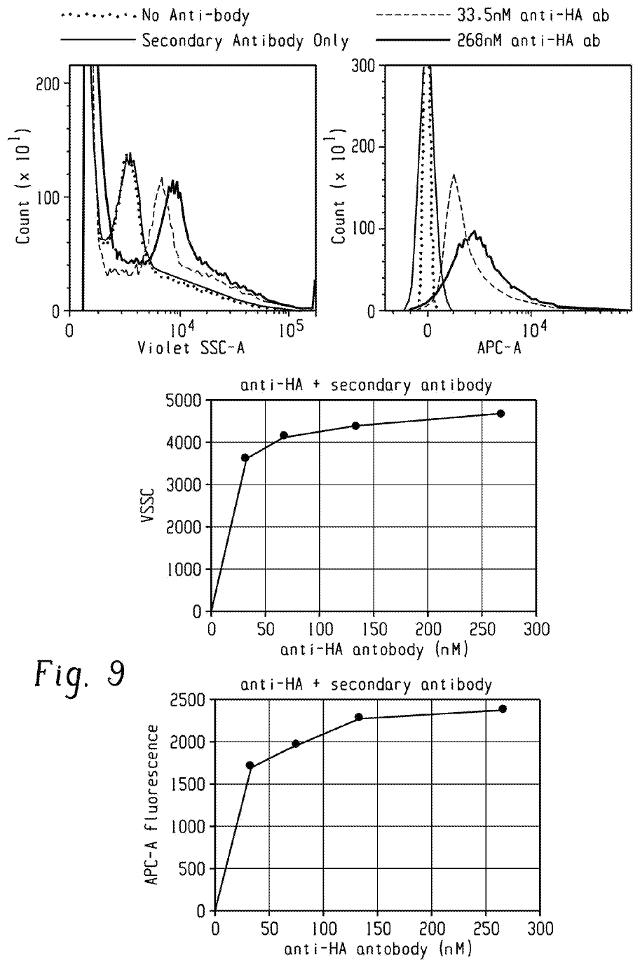

As shown in FIG. 9, antibody binding to virus particles causes a corresponding shift in VSSC. If the antibody incorporates a fluorescent molecule, antibody binding could additionally be detected using fluorescence (APC channel). Both VSSC and APC fluorescence show similar binding curves. This tweak to the method can have a range of applications in vaccine studies (e.g., evaluation), vaccine development, drug studies (evaluation), drug development, and the like. The method can be used to determine viral protein incorporation into virions under different infection conditions or to measure serum or purified antibody binding to authentic virus particles.

### Example 5: Shape/Size Distribution Based on VSSC From Virus Particles Bound by Fluorescent Antibodies

FIGS. 10A and B demonstrate that measuring the shape/size distribution based on VSSC from virus particles bound by fluorescent antibodies, and detected based on fluorescence (FIG. 10B), yields the same relative size distribution as that from unbound or antibody-bound virus particles, and detected based on VSSC (FIG. 10A). This set of experiments shows the proof of principle and illustrates a procedure/conditions for determining virus particle size distribution from “dirty”, e.g., unpurified samples deriving from cell extracts, infected animals, or infected human patients. Since cell or animal components are not fluorescent, virus particles can be pulled out of any background by virtue of using virus-specific antibodies and fluorescent secondary detection, then VSSC off of virus particles can be measured specifically to determine particle shape/size/composition/count, and the like.

This experiment used unlabeled primary antibody at saturating concentration binding to viral surface protein (in this case hemagglutinin or HA) and secondary antibody conjugated to a quantum dot (QD) binding the primary antibody. After antibody binding, the unbound antibody can be removed by gel filtration (on qEV columns). Passing virus samples on qEV columns results in minimal virus particle loss and preserves shape distributions (VSSC shift).

In FIG. 10A, particle detection/trigger is VSSC area (VSSC-A). Gate p1 is defined and captures virus particles, which are then analyzed for fluorescence or VSSC height. Before adding fluorescent secondary, virus samples are negative for fluorescence and display the same shape profile based on VSSC-height (VSSC-H)—~20% filamentous viruses.

In FIG. 10B, particle detection/trigger is either VSSC-A (first 2 columns) or fluorescence (3rd column) and samples are analyzed before or after qEV purification away from unbound antibodies. Derived shape profile (VSSC-A or VSSC-H) is the same for virus samples independent of the specific trigger (VSSC-A or fluorescence). Fluorescent antibodies have to be removed from sample by gel filtration (on qEV) to enable meaningful fluorescent triggering.

### Example 6: Influenza Viruses Exhibit a Shared Yield-Shape Trend

As shown in FIG. 11A, the genome of pleomorphic viruses encodes for a probabilistic assembly mechanism giving rise to a distribution of virion shapes, from small spherical to orders-of-magnitude longer filamentous ones. As shown in FIG. 11B, parallel infections of two cell lines were performed by several virus variants (see under Virus Strains in the Methods) under different infection conditions, and the resulting supernatants (n=40) were analyzed for virion yield and shape distributions (FIG. 10B, left panel, blue). This experiment revealed a striking shared trend: below a threshold value, the lower total particle yields were associated with a greater fraction of filamentous virions. This is strong evidence that virion shape distribution can be dynamically controlled, and contrary to common assumptions, is not genetically fixed. This insight reveals a novel aspect of phenotypic diversity: the notion of ‘tunable assembly’, i.e., dynamic regulation of virion assembly according to ongoing infection conditions. Without being held to theory, it is hypothesized that tunable assembly adds a powerful new layer to the ability of viruses to respond to changing evolutionary pressures. It is further hypothesized that that the majority of viral or cellular determinants reported to affect virion shape do so indirectly by affecting viral replication. As a critical next step, the methods described herein can be used to screen for compounds that will directly target the mechanism of virion shape regulation and decouple virus yield and shape trends (FIG. 11B). Specifically, the methods described herein allow one to identify compounds that will inhibit filamentous virion production even when the overall viral replication/production is low (FIG. 11B, left panel, magenta). Preventing filamentous virion assembly under the conditions that reduce virus replication (e.g., immune/drug pressure or suboptimal entry into cells of a new host) would eliminate the source of phenotypic diversity that might enable adaption. As shown in FIG. 11B, right panel, the size of the spherical virion subpopulation remains constant, probably defined by the size of the packaged viral genome. Both the proportion of filamentous virions and their size increases as the particle production decreases.

In this example, the targets for the small-molecule screen are two influenza mutants with defects in either cell entry or virus budding/release with lower overall virion yields and greater filamentous virion fraction (values that fall on the established trend line in FIG. 10B). The choice of two mutants to serve as screen targets includes: 1) mutants that yield a large filamentous fraction are needed to facilitate identification of compounds that inhibit filamentous-virion assembly; 2) mutants whose yield-shape properties fall on the established trend line are likely not assembly but replication mutants suggesting that their assembly machinery is unaltered by the mutations; 3) the inclusion of two mutants in separate viral proteins/functions will select for general and not mutant-specific compounds. In another strategy, two divergent WT viruses will be used as the screen targets, but infections will be performed under such a condition that reduces viral yields favoring filamentous virion production (FIG. 12). The screen further includes a WT virus control with high virion yields and low filamentous fraction, and a positive-control mutant virus with dysregulated virion assembly where lower overall yields are not associated with the greater fraction of filamentous virions (FIG. 11B-C).

In this example, inhibitors of virus entry, replication, budding or release, such as NAI, HA-targeting antibodies, M2-targeting antibodies or inhibitors, or viral RNA capping and replication inhibitor were added to infected MDCK-Siat1 or Calu3 cells after virus cell entry and the virus particles released in the supernatant after a single-cycle of infection were analyzed by flow virometry. The inhibitors affect the released particle shape distribution but in a way predicted by the newly established shape-yield trends described herein (FIGS. 11 and 12). This effect strongly argues against direct regulation of virus particle morphology by these treatments, and suggests an indirect mechanism resulting from their effects on virus entry, replication, assembly or release. These compounds will serve as negative controls or inducers of filamentous morphology in the screen for those compounds that both decrease virus particle yield and decrease the filamentous virus particle fraction, or those compounds that decrease the filamentous virus particle fraction without increasing virus particle yield.

Importantly, the data in FIG. 11C forms a genetic basis demonstrating that it is possible to shift yield vs. shape trends to disfavor filamentous virion production. Successful inhibitors will accomplish the same outcome without altering virus genetics. In addition, a screen for inhibitors may include further assay steps such as determining the effect of the potential inhibitor on virus yield in addition to shape in order to screen out inhibitors that simply affect yield but not the relative ratio of spherical vs. filamentous virus production.

The data in FIGS. 12A and 12B: 1) builds upon the model presented in FIG. 1 to demonstrate that viruses can tune shape in response to external pressures. Shape is phenotypically tunable. When a virus replicates under conditions that attenuate its entry, gene expression/replication, or assembly, and consequently reducing virion yields, virus assembly favors production of filamentous shapes. So shape is actively/phenotypically tunable, and thus shape is a more powerful evolutionary strategy than implied by the prevailing models assuming shape distribution is a fixed characteristic of a given viral genome; and 2) establishes a set of conditions that can be used to attenuate virus replication, in turn favoring filamentous virion production, and which to use in the screen for (small-molecule) inhibitors that inhibit filamentous virion production.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of determining a distribution and/or quantification of pleomorphic virus particles in a sample, comprising
detecting light scatter off of the pleomorphic virus particles and/or fluorescence of the pleomorphic virus particles, and
determining, based on the detecting, the distribution and/or quantification of spherical virus particles, irregular virus particles and/or filamentous virus particles in the sample.

2. The method of claim 1, wherein the detecting comprises near UV detection (180-400 nm), 405 nm laser light violet light side scatter (VSSC), detecting a fluorescent label on the virus particles, or a combination thereof.

3. The method of claim 1, wherein detecting comprises the use of flow virometry or microfluidics.

4. The method of claim 1, wherein the sensitivity is at least 10⁷ virus particles/ml.

5. The method of claim 1, wherein the sample is a supernatant from an infected cell.

6. The method of claim 1, wherein the sample is undiluted or treated only by dilution prior to detecting.

7. The method of claim 1, in the form of a high-throughput assay.

8. The method of claim 1, further comprising adding a fixed amount of fluorescent beads to the sample prior to detecting, and obtaining a ratio of total virus particles, spherical virus particles and/or filamentous virus particles to beads.

9. The method of claim 1, wherein a defined flow rate is used, and the method comprises determining the absolute concentrations of the spherical virus particles and/or the filamentous virus particles in the sample.

10. The method of claim 2, further comprising adding an antibody specific for a viral surface protein to the sample, wherein a shift in the VSSC is proportional to an amount of the viral surface protein in the virions at saturating antibody concentrations.

11. The method of claim 10, wherein the antibody comprises a fluorescent label, or wherein the method further comprises contacting the sample with a fluorescent secondary antibody that binds the antibody specific for viral surface proteins.

12. (canceled)

13. The method of claim 1, wherein the pleomorphic virus comprises an Orthomyxovirus, an Orthopneumovirus, a Paramyxovirus, a Filovirus, or a Coronavirus.

14. The method of claim 1, wherein the pleomorphic virus comprises an influenza virus, respiratory syncytial virus, measles virus, Ebola virus, Nipah virus, Marburg virus, severe acute respiratory syndrome coronavirus (SARS-COV), Middle East respiratory coronavirus (MERS-COV) and SARS-COV-2, or Hendra virus.

15. (canceled)

16. A method of screening a test drug, comprising
adding the test drug to a sample comprising pleomorphic virus particles or cells infected with a pleomorphic virus,
detecting light scatter off of the pleomorphic virus particles and/or fluorescence of the pleomorphic virus particles,
determining, based on the detecting, the distribution and/or quantitation of spherical particles, irregular particles, and/or filamentous particles in the sample in the presence of the test drug, and
comparing the distribution and/or quantitation in the sample with the test drug to a control distribution and/or quantitation with a control drug or no drug.

17. The method of claim 16, wherein the detecting comprises 405 nm laser light violet light side scatter (VSSC), detecting a fluorescent label on the virus particles, or a combination thereof.

18. The method of claim 16, wherein detecting comprises the use of flow virometry or microfluidics.

19. The method of claim 16, wherein the test drug is an antibody or a small molecule drug.

20. (canceled)

21. The method of claim 16, further comprising determining a number of spherical particles and/or a number of filamentous particles in the presence and absence of the test compound.

22. The method of claim 16, further comprising determining the effect of the test compound on virus assembly, virus budding, and/or virus particle release.

23. The method of claim 16, wherein the pleomorphic virus particles are from a mutant virus or viral strain.

24. (canceled)

25. A method of treating a viral infection, comprising administering to a subject in need thereof an inhibitor of filamentous particle budding or a modulator of virus morphology determined by the method of claim 16.

26. (canceled)
